(19) United States
(12) Patent Application Publication
(10) Pub. No.: US 2005/0027350 A1
(43) Pub. Date: Feb. 3, 2005

(54) Endovascular implant for the injection of an active substance into the media of a blood vessel

(75) Inventors: Carsten Momma, Rostock (DE); Andreas Becker, Berlin (DE); Robert Schmiedl, Bamberg (DE); Bernd Heublein, Hannover (DE)

Correspondence Address: HAHN LOESER & PARKS, LLP, One GOJO Plaza, Suite 300, AKRON, OH, 44311-1076, US

(73) Assignee: Biotronik Mess-und Therapiegeraete GmbH & Co Ingenieurbuero Berlin

(21) Appl. No.: 10/630,355

(22) Filed: Jul. 30, 2003

(51) Int. Cl. A61F 2/06

(52) U.S. Cl. 623/1.42; 424/426

(57) ABSTRACT

The invention concerns an endovascular implant for the application of an active substance into the media 22 of a blood vessel and two processes for the production thereof. A base body 42 of the implant has at least in portion-wise manner at a surface 40 which is towards the blood vessel, a plurality of microdevices 10 for injection of the active substance. Each microdevice 10 includes on the one hand at least one microcannula 38 which is raised out of the surface 40 of the implant to such an extent that, when the implant bears against a wall 12 of the blood vessel in surface contact, the microcannula penetrates into the media 22 of the blood vessel, and on the other hand at least one active substance deposit 36 which is in communication with at least one microcannula 38.

The invention concerns an endovascular implant for the application of an active substance into the media of a blood vessel and two processes for the production of such an implant.

### BACKGROUND OF THE ART

One of the most frequent causes of death in Western Europe and North America is coronary heart disease. According to recent knowledge, in particular inflammatory processes are the driving force behind arteriosclerosis. The process is supposedly initiated by the increased deposit of low-density lipoproteins (LDL-particles) in the intima of the vessel wall. After penetrating into the intima the LDL-particles are chemically modified by oxidants. The modified LDL-particles in turn cause the endothelium cells which line the inner vessel walls to activate the immune system. As a consequence monocytes pass into the intima and mature to macrophages. In conjunction with the T-cells which also enter inflammation mediators such as immune messenger substances and proliferatively acting substances are liberated and the macrophages begin to receive the modified LDL-particles. The lipid lesions which are formed from T-cells and the macrophages which are filled with LDL-particles and which by virtue of their appearance are referred to as foam cells represent an early form of arteriosclerotic plaque. The inflammation reaction in the intima, by virtue of corresponding inflammation mediators, causes smooth muscle cells of the further outwardly disposed media of the vessel wall to migrate to under the endothelium cells. There they replicate and form a fibrous cover layer from the fiber protein collagen, which delimits the subjacent lipid core of foam cells from the bloodstream. The deep-ranging structural changes which are then present in the vessel wall are referred to in summary as plaque.

Arteriosclerotic plaque initially expands relatively little in the direction of the bloodstream as the latter can expand as a compensation effect. With time however there is a constriction in the blood channel (stenosis), the first symptoms of which occur in physical stress. The constricted artery can then no longer expand sufficiently in order better to supply blood to the tissue to be supplied therewith. If it is a cardiac artery that is affected, the patient frequently complains about a feeling of pressure and tightness behind the sternum (angina pectoris). When other arteries are involved, painful cramps are a frequently occurring sign of the stenosis.

The stenosis can ultimately result in complete closure of the blood stream (cardiac infarction, stroke). Recent investigations have shown however that this occurs only in about 15 percent of cases solely due to plaque formation. Rather, the progressive breakdown of the fibrous cover layer of collagen, which is caused by certain inflammation mediators from the foam cells, seems to be a crucial additional factor. If the fibrous cover layer tears open the lipid core can come directly into contact with the blood. As, as a consequence of the inflammation reaction, tissue factors (TF) are produced at the same time in the foam cells, and these are very potent triggers of the coagulation cascade, the blood clot which forms can block off the blood vessel acutely completely (acute infarction) or partially (unstable angina pectoris).

Non-operative stenosis treatment methods were established more than twenty years ago, in which inter alia the blood vessel is expanded again by balloon dilation (PTCA—percutaneous transluminal coronary angioplasty). It will be noted however that expansion of the blood vessel gives rise to injuries (tears, so-called dissections) in the vessel wall, which admittedly predominantly heal without any problem but which in about a third of cases, due to triggered cell growth, result in growths (proliferation) which ultimately result in renewed vessel constriction (restenosis). The expansion effect also does not eliminate the physiological causes of the stenosis, that is to say the changes in the vessel wall. A further cause of restenosis is the elasticity of the expanded blood vessel. After the balloon is removed the blood vessel contracts excessively so that the vessel cross-section is reduced (obstruction, referred to as negative remodeling). The latter effect can only be avoided by the placement of a stent. The use of stents admittedly makes it possible to achieve an optimum vessel cross-section, but the use of stents also results in very minor damage which can induce proliferation and thus ultimately can trigger restenosis.

In the meantime extensive knowledge has been acquired in regard to the cell-biological mechanism and to the triggering factors of stenosis and restenosis. As already explained above restenosis occurs as a reaction on the part of the vessel wall to local damage as a consequence of expansion of the arteriosclerotic plaque. By way of complex active mechanisms lumen-directed migration and proliferation of the smooth muscle cells of the media and the adventitia is induced (neointimal hyperplasy). Under the influence of various growth factors the smooth muscle cells produce a cover layer of matrix proteins (elastin, collagen, proteoglycans) whose uncontrolled growth can gradually result in constriction of the lumen. Systematically medicinal therapy involvements provide inter alia for the oral administration of calcium antagonists, ACE-inhibitors, anti-coagulants, anti-aggregants, fish oils, anti-proliferative substances, anti-inflammatory substances and serotonin-antagonists, but hitherto, significant reductions in the restenosis rates have not been achieved in that way.

A basic problem in terms of medicinal treatment or prevention of re-stenosis is the in part considerable side-effects of the active substances. The attempt is made to circumvent these inter alia by application which is locally delimited to the greatest possible extent, in suitably low doses. The so-called concept of local drug delivery (LDD) provides that the active substance or substances is or are liberated directly at the location of the occurrence and limited to that area. For that purpose, a surface of the endovascular implant, that is to say in particular a stent, which faces towards the vessel wall, is frequently provided with an active coating. The active component of the coating in the form of a therapeutic active substance can be bound directly to the surface of the implant or embedded in a suitable drug carrier. In the latter case the active substance is liberated by diffusion and possibly gradual breakdown of the biodegradable carrier.

U.S. Pat. No. 6,287,628 proposes an implant and a process for the production thereof, in which the active substance is not applied directly to the surface of the implant but is provided in an active substance deposit. The active substance deposits are introduced in the form of cavities in the base body of the implant and arranged on the outside of the implant. That is intended to prevent the active substance from being excessively flushed out by the continual flow of blood.

In addition, U.S. Pat. No. 6,254,632 shows structures which project out of the plane of the implant surface and which are intended inter alia to furnish the active substance. The structures in crater form are intended to promote delivery of the substance directly to the wall of the vessel. The shape and in particular the height of the structure—according to the information supplied they are in the range of between 10 and 80 μm—are so predetermined that the structure does not penetrate into the wall of the vessel but at most deforms it.

In all the known structures the surface of the implant bears directly against the intima of the blood vessel. Accordingly the liberated active substance firstly has to pass through that interface by diffusion. Diffusion of the active substance can however be impeded by generally present plaque, calcification or thickened vessel wall layers. It is precisely in relation to coronary diseases that studies have shown that the intima is markedly increased in its wall thickness and can be over 150 μm in width. In addition considerable amounts of the active substance can be entrained through the constantly occurring lumen flow in the blood vessel as long as the active substance has not penetrated into the wall of the vessel. For certain medicinal-therapeutic effects (for example gene therapy) it is necessary to get to or into the proximity of the smooth muscle cells of the wall of the vessel (media region) as quickly as possible and with a high level of concentration.

Therefore the object of the present invention is to provide an endovascular implant which overcomes the above-depicted disadvantages of the state of the art and permits the application of very small amounts of active substance in a locally delimited region, in particular directly into the media of the vessel wall.

### SUMMARY OF THE INVENTION

That object is attained by the endovascular implant for the application of an active substance into the media of a blood vessel, having the features recited in the appended claims, and the associated production processes as set forth in the claims. For that purpose, at least in portion-wise manner at a surface which is towards the wall of the vessel, the base body according to the invention of the implant has a plurality of microdevices for the application of the active substance. Each microdevice includes:
at least one microcannula which is raised out of the plane of the surface of the implant to such an extent that when the implant bears in surface contact against the wall of the blood vessel the microcannula penetrates into the media of the blood vessel, and
at least one active substance deposit which is in communication with at least one microcannula.

The above-indicated configuration of the implant according to the invention makes it possible for the active substances to be eluted directly at the specific location of the action of the active substances, namely the media. Concomitantly therewith the applied amount of active substance can be substantially reduced so that the production costs of the LDD-implant can be markedly reduced and alocal side-effects are very substantially excluded.

In accordance with a preferred configuration of the invention the microcannulae project approximately 100-400 μm out of the surface of the implant. This therefore ensures that they pierce the intima, but do not yet reach vessel regions at greater depth, that is to say the adventitia. In the case of intracoronary use, by virtue of the anatomical particularities of the vessel walls, microcannula lengths of 150-300 μm, in particular 180-250 μm are preferred, as in the vast majority of cases they afford an optimum depth of penetration of the microcannulae. In the individual case however the thickness of the vessel wall may differ markedly for, besides the factors involved in diseases, individual variations in vessel wall thicknesses are also found. In the meantime diagnostic systems have been developed (for example intravascular ultrasound) which make it possible to measure the thickness of the intima so that additional diagnostic equipment is available for the doctor carrying out the treatment, for the selection of an implant with a microcannula length of sufficient size.

The microdevice can be on the one hand an integral component part of the base body of the implant, that is to say it can be formed therefrom by suitable processing or machining steps. On the other hand however the microdevice can also be embodied in the form of a structure which is fitted on the base body (hybrid technology).

In the former case the implant is preferably produced in such a way that a cavity is introduced into the metallic base body of the implant by partial removal of material on the surface of the implant, that faces towards the wall of the vessel. That can be effected for example by having recourse to tried-and-tested laser machining processes which are mostly also applied when cutting out the stent structures. The cavity which is a few micrometers deep is covered in a subsequent step in the process with an insulating material, wherein the material is applied in such a way that it slightly overlaps the peripherally extending edge of the cavity. Thereafter, the material of the base body is removed by electropolishing in the region of the surface, which is not covered by the insulating material, in which case the desired microcannulae are gradually formed above the cavity. After the electropolishing operation is concluded the insulating material is removed with a suitable solvent. Accordingly the process only modifies per se known processing steps in the production of stents so that it is possible to have recourse to existing resources of experience.

If the microdevices are to be produced using the hybrid technology in the form of independent structures on the surface of the implant, it is possible to have recourse to per se known rapid prototyping processes for polymer deposition and for sintering. Thus for example cross-linking of monomers to form polymers can be induced by micro-stereolithography, under the action of light. For that purpose a laser scanning unit exposes a defined area on the surface of the liquid monomer, in a hatching-like pattern, and in that way, with a given depth of penetration, hardens a layer of the pattern to be produced. A displacement unit in the z-direction provides that the substrate is lowered layer by layer by the defined layer thickness or the laser focus is raised. In the following processing step, the monomer, over the previously produced solid layer, is caused to polymerise by exposure and that processing step is repeated until the desired structure is produced. After the layer generation operation, remaining monomer residues are removed by suitable solvents. The procedures are monitored by complex control mechanisms and permit the production of microstructures of micrometer dimensions which were produced previously with common programs in the form of for example a CAD-original. At any event the microdevices produced by rapid prototyping processes contain an active substance deposit which is joined to at least one microcannula.

In a variant of the invention, a liberation behaviour in respect of the at least one deposited active substance is established in such a way that the at least one active substance is liberated only after penetration of the microcannulae into the media of the blood vessel. Uncontrolled elution is to be avoided in that way and the total dose of the active substance is to be further reduced. Thus the microdevice, after introduction of the at least one active substance into the active substance deposit, can be closed by a cover layer comprising a biodegradable material which is completely broken down only after penetration into the media. It is further possible for the at least one active substance to be embedded in a biodegradable drug carrier. In accordance with this variant, it is also possible for the liberation behaviour of the active substance to be influenced by way of the degradation behaviour of the carrier. It will be appreciated that both these proposed procedures can be combined together, that is to say liberation is controlled in the manner in accordance with the invention by a suitable cover layer and a drug carrier.

It is further preferred that a plurality of active substances are provided in the active substance deposit and the liberation thereof takes place in a stepwise manner. That can be effected for example in such a way that a plurality of layers of biodegradable drug carriers with various active substances are introduced in layer-wise fashion into the active substance deposit. The layers are successively broken down from the outside inwardly, and accordingly successively liberate the various active substances. It is also possible for one or more separating layers—again comprising a biodegradable material—to be introduced into the active substance deposit. The separating layers serve to separate the various active substances and are broken down in succession. A time-staged process of that kind makes it possible to effectively influence the underlying mechanisms of restenosis. Thus for example the initial mechanisms of restenosis can be specifically and targetedly combated by anti-proliferative substances and at a later time, by the application of anti-inflammatory substances and the like, cell migration can be prevented.

Preferably, the regions of the surface of the implant, which are outside the microdevice, are also covered with a layer of a biodegradable material. More specifically, it has been found that bioactive surfaces of that kind markedly reduce the restenosis rate. Preferred materials are hyaluronic acid polymer, polylactides and heparin.

If the layer terminates flush in the peripheral direction with a tip of the microcannula of the microdevice or if it is indeed completely covered then the structure on the one hand is initially protected from mechanical damage upon being introduced into the body while on the other hand penetration of the microcannula into the vessel wall can be controlled, in respect of time. The microcannulae slowly penetrate through the intima into the media, due to the gradual breakdown of the layer. That slow penetration prevents damage to the vessel wall, which damage in turn could be the starting point for restenosis processes. In order to prevent premature liberation of the active substance, the liberation behaviour of the deposited active substance must be suitably matched by virtue of the choice of the biodegradable drug carrier or a cover layer. Hyaluronic acid is particularly suitable for coating the surface in the regions outside the microstructure. A breakdown behaviour on the part of the hyaluronic acid can be established by specific and targeted cross-linking in the desired manner. Therefore, that material is also suitable as the cover layer and the drug carrier. The production of cross-linked hyaluronic acid coatings and the influencing of degradation behaviour is known in principle from the state of the art. In general terms, the degradation time increases with an increasing degree of polymerisation and/or degree of cross-linking of the carrier. An elution characteristic which applies in respect of the embedded active substance depends on the degree of polymerisation and cross-linking, besides depending on diffusion processes. In general elution is increased in length, with an increasing degradation time.

It is further preferred if the implant is a stent, in particular a coronary stent, for it is precisely in relation to those implants that the risk of restenosis is high. In addition the stent preferably has self-expanding structures which promote gradual penetration of the microcannula into the vessel wall. That is particularly desirable if, as described above, the microdevices are covered with a protective layer (covering). It will be appreciated that it is also possible to provide for penetration of the microcannulae, by means of balloon dilation.

In a further variant of the invention, the base body of the implant, in particular the stent, is also formed from a biodegradable magnesium alloy. Complete breakdown of the stent provides for long-term elimination of the factors which possibly trigger off restenosis.

Further preferred configurations of the invention will be apparent from the other features recited in the appendant claims.

### BRIEF DESCRIPTION OF THE DRAWINGS

The invention is described in greater detail hereinafter by means of an embodiment and with reference to the drawings in which:

FIG. 1 is a diagrammatic view in cross-section through an endovascular implant in the region of a microdevice and after penetration into a vessel wall,

FIGS. 2-5 are diagrammatic views in cross-section through microdevices in which the active substance is provided in accordance with four alternatives,

FIG. 6 shows the microdevice with an additional coating surrounding it,

FIG. 7 shows the microdevice with an additional coating surrounding it as a protective layer (covering),

FIGS. 8a-8d show diagrammatic views of a production process for the microdevice in accordance with a first variant,

FIG. 9 is a view showing the principle of the rapid prototyping process with which production of the microdevice can be implemented in accordance with a second variant, and

FIGS. 10a-10b are diagrammatic views of two microdevices which were produced using the rapid prototyping process on an implant surface.

### DETAILED DESCRIPTION OF A PREFERRED EMBODIMENT

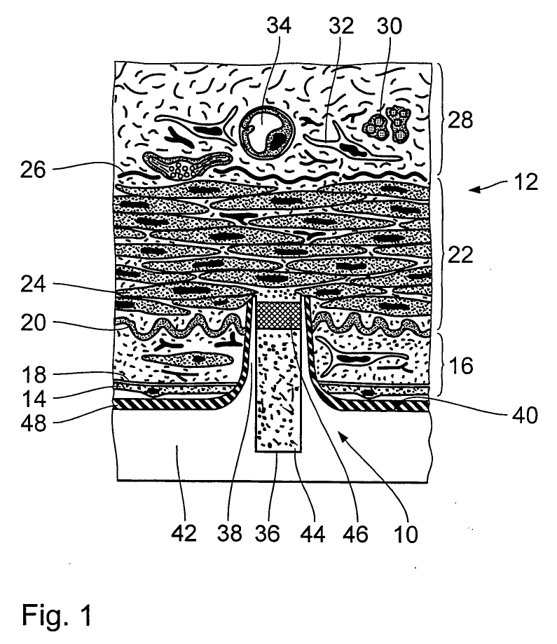

FIG. 1 diagrammatically shows a view in cross-section through an endovascular implant in the region of a microdevice 10 which has already penetrated into a vessel wall 12 of a muscular artery. The endovascular implant can be in particular a stent. The stent is formed from a biocompatible material, for example nitinol, medical steel, tantalum, platinum-iridium alloys, gold or the like. It is also possible to use a biodegradable magnesium alloy. The design and dimensioning of the stent can be variable to a wide extent. They only have to permit the arrangement or the provision of the microdevices 10 on the outside surface thereof.

In accordance with prevailing histological teaching, the vessel wall 12 of the artery is divided into three layers. Following the inner endothelium cells 14 which line the vessel wall 12, there extends the region of the so-called intima 16 which is delimited by a basal lamina 18 and an inner elastic membrane 20. The intima 16 is adjoined by the media 22 which, in the case of muscular arteries, is formed from a plurality of myofibroblasts 24, that is to say smooth muscle cells. The adventitia 28 then follows, separated from the media 22 by an outer elastic membrane 26. Nerve fiber bundles 30, fibroblasts 32 and blood vessels 34 are to be found in the adventitia 28.

Upon damage to the vessel wall 12—whether it is due to balloon dilation or also when a stent is being fixed in the desired position—mechanisms causing restenosis can be triggered off. In particular the media 22 and the adventitia 28 are involved in the processes underlying restenosis, either as an initiator in respect of proliferation by the liberation of inflammation mediators or as the location of origin of cell migration in a case involving neointima proliferation in which the myofibroblasts 24 form fibrous cover layers in the region of the intima 16, which is towards the bloodstream. Effective combating of restenosis therefore has to take place in particular in those regions.

For that purpose the stent has the microdevice 10 which projects through the intima 16 into the media 22 and which there can liberate a therapeutic active substance in a manner which is going to be discussed in greater detail hereinafter. The microdevice 10 comprises an active substance deposit 36 and at least one connected microcannula 38. As illustrated, the two components can blend into each other. In order actually also to reach the location of the action involved, namely the media 22, the microcannula 38 projects by between 100 and 400 μm out of the surface 40 of the stent, which faces towards the vessel wall 12. For intracardial use, microcannula lengths of between 150 and 300 μm, in particular between 180 and 250 μm, have proven to be particularly effective as they can assume the desired position in far above 90% of cases. The length must be such that any plaque which is possibly present in the intima 16 can be penetrated. A diameter for the microcannula 38 is between about 20 and 200 μm.

The number and position of the microdevices 10 on the stent can be adapted to the respective requirements involved and is substantially dependent on the amount of the active substance to be eluted and in particular the stent design. In general terms, the aim is to provide that the microdevices 10 are distributed over the surface 40 in as homogeneous a fashion as possible and over the largest possible surface area, in order to ensure uniform dosage of the active substance. As the diffusion processes in the media 22 take place relatively slowly, the number of microdevices 10 on the peripheral surface of the stent should be approximately of the order of magnitude of between 2 and 20 per cm².

In the present example, the active substance deposit 36 and the microcannula 38 are completely incorporated into a base body 42 of the stent. It is also possible for the microdevice 10 to be produced using the hybrid technology, that is to say in the form of a structure which is delimited from the base body 42. Two possible ways in which production can be effected in practice are described in greater detail hereinafter.

An active substance—indicated here by a layer 44—is introduced into the active substance deposit 36 of the microdevice 10. The active substance deposit 36 initially remains closed by a cover layer 46 comprising a biodegradable material. The degradation behaviour of the cover layer 46 is so established that liberation of the active substance can take place only after complete penetration into the media 22. The surface 40 of the stent is still covered with an also biodegradable additional layer 48, the function of which will be described in greater detail hereinafter.

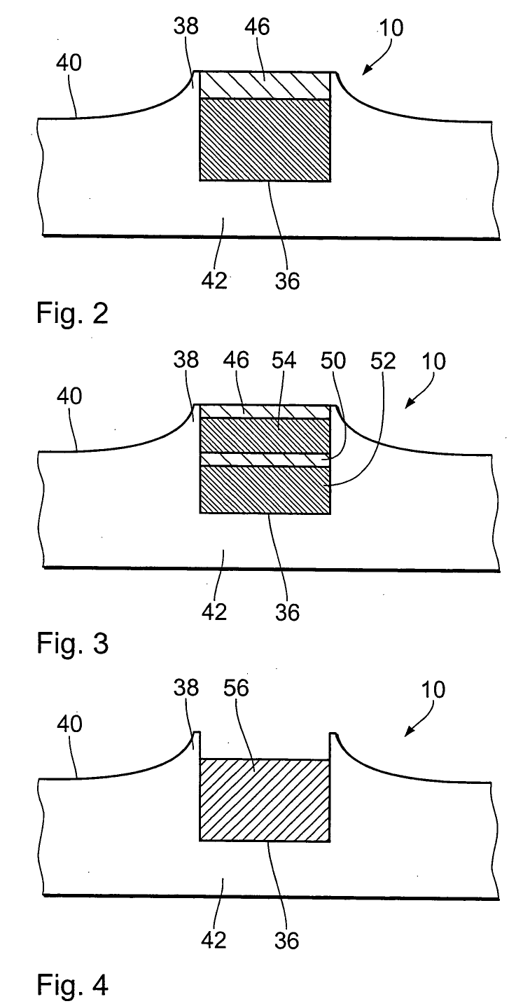

FIG. 2 shows a first variant of the way in which the liberation behaviour of the active substance can be influenced in the desired manner. For that purpose the active substance is introduced into the active substance deposit 36 in its preferred pharmaceutical form of application. The active substance deposit 36 is then closed by the bioresorbable cover layer 46.

If a plurality of active substances are to be liberated in succession, then—as diagrammatically indicated in FIG. 3—besides the cover layer 46 the arrangement may have a further separating layer 50 in the active substance deposit 36. The separating layer 50 divides the volume of the active substance deposit 36 into two regions 52, 54, in each of which there is a respective active substance in its pharmaceutically desired form of application.

FIG. 4 shows a further alternative embodiment for resolving the problem of liberation of the active substance. The active substance is embedded in a suitable drug carrier and is introduced in the form of a homogeneous bioresorbable layer 56 into the active substance deposit 36. The active substance is gradually liberated by progressive breakdown of the bioresorbable layer 56.

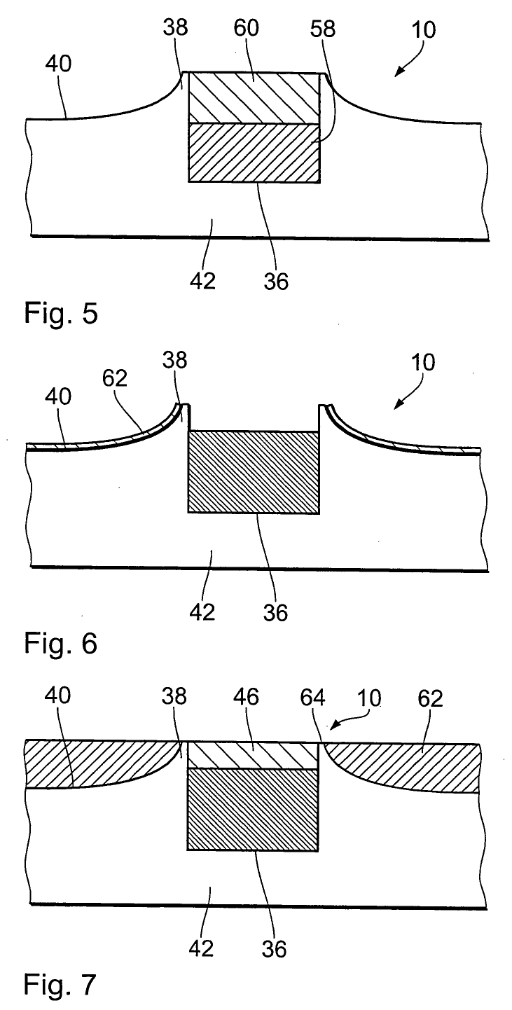

In accordance with a further variant, it is also possible for a plurality of layers of bioresorbable drug carriers with optionally different active substances to be introduced into the active substance deposit 36. FIG. 5 shows by way of example two such layers 58 and 60. The layer 58 contains an active substance which is to be applied into the media 22 only at a later moment in time while the upper layer 60 contains an active substance which is intended to inhibit the restenosis-triggering mechanisms as early as possible. For that purpose for example a first active substance which prevents the liberation of inflammation mediators could be introduced into the layer 60 and a second active substance for influencing cell migration could be introduced into the layer 58. It is also possible, by the introduction of a plurality of layers, to establish a temporary dose of an individual active substance or a combination of active substances, insofar as the layers have different contents of the active substance or combination of substances. Likewise, a variation in the degradation behaviour of the individual layers in the active substance deposit can be used to influence the temporary dose.

FIG. 6 diagrammatically shows a further alternative form of providing the active substance in the microdevice 10, in which the surface 40 of the stent is covered with an additional layer 62 of a bioresorbable material. The additional layer 62 serves to suppress inflammatory processes and, as shown in FIG. 7, can adjoin an upper tip 64 of the microcannular 38, in flush relationship therewith, or can even completely cover over the entire microdevice 10. That provides that, in the implantation procedure, the stent does not damage the vessel wall 12 by virtue of the microdevices 10, or the microdevices 10 are not mechanically damaged (protective covering).

With the progressive breakdown in the additional layer 62 the microcannula 38 penetrates through the intima 16 into the media 22. That process can be assisted by self-expanding structures in the stent. For example a shape memory alloy such as nitinol can be used for that purpose. It is however also possible to envisage manual dilation with balloon catheters at given time intervals. In order to prevent premature liberation of the active substance, the degradation behaviour of the cover layer 46 is so adapted that the breakdown thereof is concluded only after complete breakdown of the additional layer 62 and thus the microcannula 38 has, in all probability, already reached the location of action, namely the media 22 of the vessel wall 12.

Suitable bioresorbable materials for the above-mentioned cover layers, separating layers, additional layers and drug carriers are in particular polycaprolactones (PCL), poly-D,L-lactic acids (DL-PLA), poly-L-lactic acids (L-PLA), polyhydroxybutyrates, polydioxanones, glycolidic polylactides, polyorthoesters, polyanhydrides, polyglycolic acids and their copolymers, polyphosphoresters, polyamino acids, polytrimethylene carbonate, polyphosphazenes, polyimino carbonates, aliphatic polycarbonates, heparin, fibrin, fibrinogen, cellulose, collagen, alginates, chitosans and cross-linked hyaluronic acids.

Active substances for direct application into the media 22 are in particular anti-angiogenic, anti-inflammatory and anti-proliferative therapeutically-effective substances. The anti-angiogenic substances include for example retinoic acid and derivatives thereof, suramin, metallproteinase-1- and metallproteinase-2-inhibitors, epothilone, colchicine, vinblastine and paclitaxel. Anti-inflammatory substances include for example salicylates, fenoprofen, ketoprofen, tolmetin, cortisone, dexamethosone, cyclosporine, azatidine, rapamycin, tacrolimus, everolimus and diphenylimidazole. Anti-proliferative therapeutic substances which are to be considered are in particular anti-estrogens and hormones (estradiol, tamoxifen, testolactone, etc) and cytotoxic agents (bleomycin, doxorubicine, idarubicine, colchicine, etc).

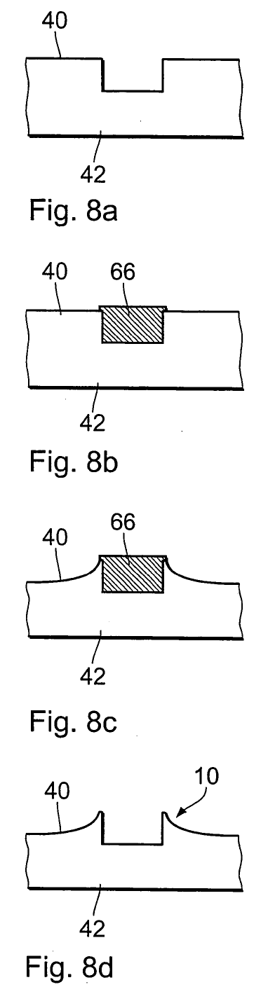

FIGS. 8a through 8d diagrammatically show a portion of a stent in which a microdevice 10 is to be produced by certain processing steps. The individual structure elements of the metallic base body 42 of the stent are usually produced by laser cutting. Suitable for this purpose are for example lasers with an operating range around 1000 nm and an output power of about 3-5 W, whose pulse length can be varied in the range of a few hundredths of a millisecond and which can be focussed on to a region of a few μm². Before, during or subsequently to cutting of the bar portions etc, a cavity can be introduced into the surface 40 of the base body 42 (see FIG. 8a) by targeted partial laser removal. It will be self-evident that the operating parameters of the laser are altered during the partial laser removal operation, in comparison with the operating parameters during the laser cutting process. The output power, pulse duration and/or duration of interaction are appropriately reduced. In this respect, the depth of the cavity should correspond at least to the desired length of the microcannula, that is to say it should be at least 100 μm. Likewise the diameter of the cavity produced predetermines a diameter of the microcannula (this is usually between 20 and 200 μm).

In a subsequent process step (FIG. 8b) the cavity and in slightly overlapping relationship also an edge portion extending around the cavity are covered with an electrically insulating material 66. The material 66 involved can be for example lacquer, wax or the like which is applied in the form of an emulsion or solution by suitable spraying procedures. Material is removed from the base body 42 by the subsequent electropolishing operation, in which case the base body 42 remains in the regions of the surface 40, which are covered by the insulating material 66, and the microcannula is gradually formed (FIG. 8c). The electropolishing operation is also routinely applied in the production of stents as it has been shown that smooth surfaces further reduce the restenosis rate. Finally the insulating material 66 is removed again with suitable solvents (FIG. 8d).

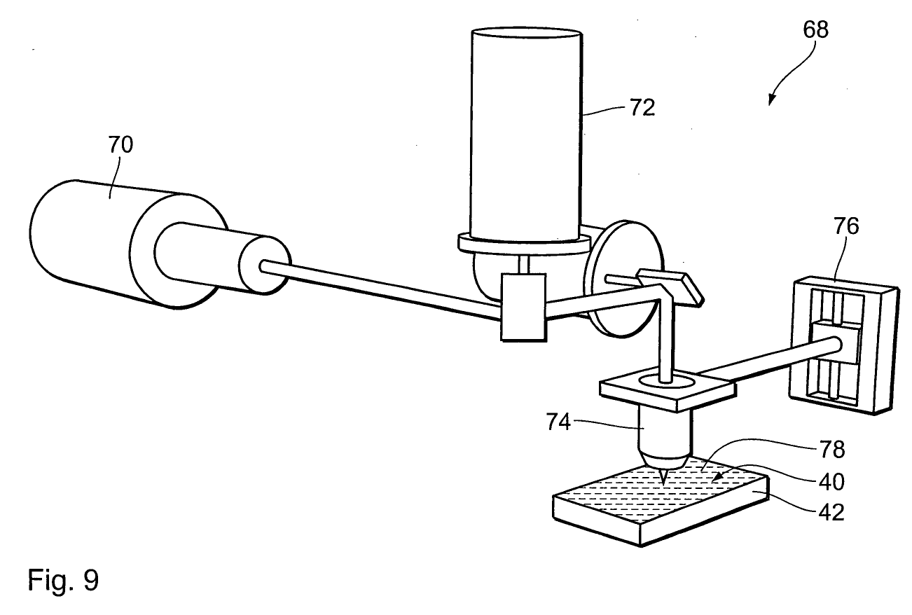

Instead of complete integration of the microdevice 10 into the base body 42 of the stent, as was described hereinbefore, the microdevice can also be applied in the form of a separate structure to the surface 40 of the implant, using hybrid technology. In the meantime, rapid prototyping processes based on microlithography steps have been developed for microstructures of that kind (see for example TÖNSHOFF, H K; KÖRBER, K; BEIL, A: Micro-rapid prototyping—a new machine system enabling micro-stereolithography and laser sintering processes. In: Proc of MICRO. tec 2000, Vol 2, 25th to 27th Sep. 2000, Hannover—ISBN 3-8007-2579-7, pages 347-42). In that case, suitable resins are polymerised by a focused laser beam which is as narrow as possible, in the exposed region. Besides the use of polymers, sintered materials can also be used. FIG. 9 shows in simplified form the structure in principle of a micro-stereolithography apparatus 68 which is suitable for carrying out the rapid prototyping process.

The microdevices 10 are built up layer by layer by polymerisation, starting from the surface 40 of the base body 42. A laser source 70 serves as a pulsed light source. The laser beam produced is fed by way of a scanner unit 22 into a strongly focusing lens 74 which is mounted movably by way of a control member 76. The surface 40 of the base body 72 is covered with a fluid monomer layer 78 which is a few micrometers thick. The laser beam exposes in a hatching-like pattern a defined area on the surface 40 in the fluid monomer and in that way in the focus region hardens a layer of the model to be produced, to a given depth of penetration. The displacement member 76 provides that the substrate or the laser focus is lowered or raised respectively, layer by layer, by the defined layer thickness, usually in micrometer steps. In the following processing step, the monomer over the previously produced solid layer is caused to undergo polymerisation by exposure (by single- or multi-photon processes) and that processing step is repeated until the desired structure is produced. After layer generation, remaining monomer residues are removed by suitable solvents. A complex control system (also not shown) monitors production by way of the scanner unit 72. The control system, on the basis of a model produced for example in CAD-format, co-ordinates the operating movements of the lens 74, the intensity and pulse duration of the laser source 70 and the movement of the elevator device. As microlithography apparatuses 68 of the illustrated kind are already sufficiently known from the state of the art, the apparatus will not be described in detail herein.

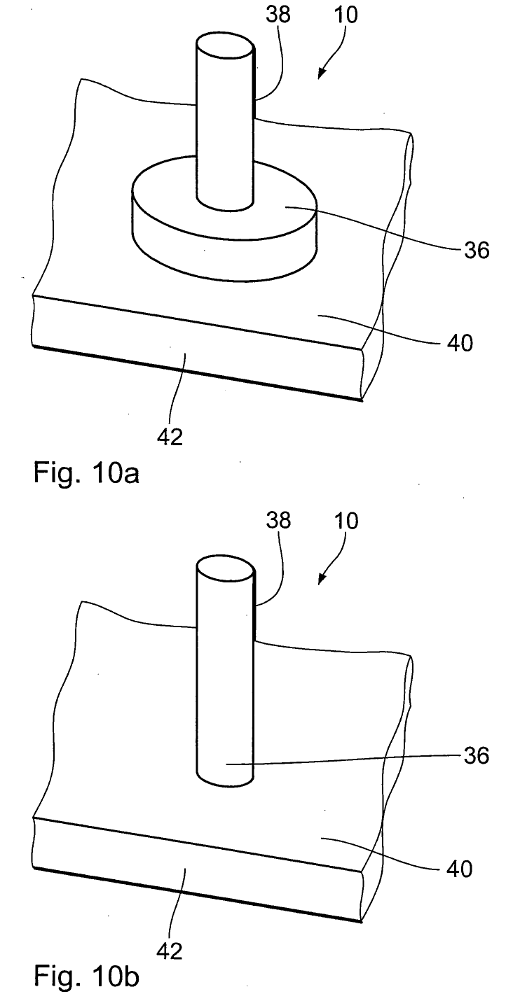

A first microdevice 10 produced by rapid prototyping processes is diagrammatically shown in FIG. 10a. The microdevice 10 includes a tubular microcannula 38 which is fitted on a cylindrical active substance deposit 36. The overall height of the structure is about 200 μm. The two main components of the microdevice 10 can also blend into each other in contour-less fashion, as shown in FIG. 10b. The tubular microdevice 10 projects about 150 μm up from the surface 40 of the base body 42. Preferably biocompatible polymers are used for the production process.

The active substance can be introduced into the prepared microdevices 10 for example by a procedure whereby the stent surface 40 is wetted with a solution of the active substance in a suitable solvent, in which case the solution also penetrates into the microdevices 10. After the drying operation the active substance is blown off in the regions outside the microdevices 10. For that purpose it is possible to use for example an air lance with some kPa blower power at an angle of about 90° and a spacing of a few centimeters. Operation can also be effected in the same manner when applying the polymers for cover layers, separating layers and drug carriers. Finally the surface 40 can be covered with a protective additional layer (protective covering), for example using an immersion or spray process.

1. An endovascular implant for applying an active substance into the media of a blood vessel, said implant comprising:
a base body which has a plurality of microdevices for applying the active substance disposed at least in portion-wise manner at a surface of the implant adapted for facing towards the blood vessel, wherein each said microdevice includes at least one microcannula which is raised out of the implant surface to such an extent that, when the implant bears in surface contact against a wall of the blood vessel, the microcannula penetrates into the media of the blood vessel, and
at least one deposit of the active substance which is in communication with at least one said microcannula.

2. The implant of claim 1, wherein:
the microcannulae are of a length of 100-400 μm.

3. The implant of claim 2, wherein:
the microcannulae are of a length of 150-300 μm.

4. The implant of claim 3, wherein:
the microcannulae are of a length of 180-250 μm.

5. The implant of claim 2, wherein:
the microcannulae are of a diameter of 20-200 μm.

6. The implant of claim 1, wherein:
the microdevices are component parts of the base body.

7. The implant of claim 5, wherein:
the microdevices are component parts of the base body.

8. The implant of claim 5, wherein:
the microdevices are applied to the base body using hybrid technology.

9. The implant of claim 1, wherein:
the microdevices are applied to the base body using hybrid technology.

10. The implant of claim 5, wherein:
a liberation behaviour in respect of the at least one active substance to be deposited is so established that the at least one active substance is liberated only after penetration of the microcannulae into the media of the blood vessel.

11. The implant of claim 1, wherein:
the at least one active substance to be deposited is liberated only after penetration of the microcannulae into the media of the blood vessel.

12. The implant of claim 10, wherein:
a cover layer of a biodegradable material closes the plurality of microdevices after the at least one active substance has been introduced into the active substance deposit.

13. The implant of claim 11, wherein:
a cover layer of a biodegradable material closes the plurality of microdevices after the at least one active substance has been introduced into the active substance deposit.

14. The implant of claim 10, wherein:
the at least one active substance is embedded in a biodegradable drug carrier.

15. The implant of claim 11, wherein:
the at least one active substance is embedded in a biodegradable drug carrier.

16. The implant of claim 10, wherein:
a plurality of active substances are introduced into the active substance deposit such that stepwise liberation of the active substances occurs.

17. The implant of claim 11, wherein:
a plurality of active substances are introduced into the active substance deposit such that stepwise liberation of the active substances occurs.

18. The implant of claim 16, wherein:
a plurality of layers of biodegradable drug carriers with embedded active substances are introduced into the active substance deposit and are successively broken down.

19. The implant of claim 17, wherein:
a plurality of layers of biodegradable drug carriers with embedded active substances are introduced into the active substance deposit and are successively broken down.

20. The implant of claim 16, comprising:
at least one separating layer of a biodegradable material, each of which is successively broken down and which separates the various active substances from each other.

21. The implant of claim 17, comprising:
at least one separating layer of a biodegradable material, each of which is successively broken down and which separates the various active substances from each other.

22. The implant of claim 1, wherein:
regions of the surface of the implant that are outside the microdevice are covered with a layer of a biodegradable material.

23. The implant of claim 22, wherein:
the layer of biodegradable material terminates flush in a peripheral direction at a tip of the microcannulae of the microdevice or completely covers the microdevice and
a breakdown behaviour on the part of the layer is matched with the liberation behaviour of the active substance, such that liberation of the active substance begins only after complete breakdown of the layer.

24. The implant of claim 22, comprising:
self-expanding structures which promote progressive penetration of the microcannulae into the vessel wall.

25. The implant of claim 22, wherein:
the layer of biodegradable material comprises hyaluronic acid polymers with different degradation kinetics.

26. The implant of claim 1, wherein:
the implant is a stent.

27. The implant of claim 26, wherein:
the stent is adapted for use as a coronary stent.

28. The implant of claim 26, wherein:
the base body is formed at least in portion-wise manner from a biodegradable material.

29. The implant of claim 28, wherein:
the base body is formed, at least in portion-wise manner, from a magnesium alloy.

30. A process for producing a microdevice for injecting an active substance into the media of a blood vessel, comprising the steps of:
a) producing a cavity on a surface of a metallic medical implant by partial material removal, the surface being adapted to face the blood vessel wall during use;
b) covering the cavity and a peripherally extending edge of the cavity are covered with an insulating material;
c) removing additional material by electropolishing in the regions of the surface that are not covered by the insulating material; and
d) removing the insulating material by suitable solvents.

31. The method of claim 30, wherein:
the cavity-producing step is achieved by laser material removal.

32. A process for producing, on an implant, a microdevice for injecting an active substance into the media of a blood vessel, comprising the step of:
building up the microdevice stepwise by a rapid prototyping process based on microlithography steps, on a surface of the implant, the surface being adapted to face the blood vessel wall during use.
